(19) United States
(12) Patent Application Publication
(10) Pub. No.: US 2006/0276788 A1
(43) Pub. Date: Dec. 7, 2006

(54) Osteoconductive spinal fixation system

(75) Inventors: Bret M. Berry, Sandy, UT (US); Ashok C. Khandkar, Salt Lake City, UT (US); Ramaswamy Lakshminarayanan, Salt Lake City, UT (US)

Correspondence Address: KELLY LOWRY & KELLEY, LLP, 6320 CANOGA AVENUE, SUITE 1650, WOODLAND HILLS, CA, 91367, US

(73) Assignee: Amedica Corporation

(21) Appl. No.: 11/139,126

(22) Filed: May 26, 2005

(51) Int. Cl. A61F 2/30

(52) U.S. Cl. 606/61

(57) ABSTRACT

An improved spinal fixation system is provided for human implantation, including a set of screws with interconnecting rods for implantation into the pedicle and between two adjacent vertebrae or a plate with screws for fixating two adjacent vertebrae. The screws, rods, and plates include a substrate portion of high strength biocompatible material and a controlled porosity analogous to natural bone. The substrate portion may be coated with a bio-active surface coating material such as hydroxyapatite or a calcium phosphate to promote bone ingrowth and enhanced bone fusion. Upon implantation, the fixation system provides a desired combination of mechanical strength together with osteoconductivity and bio-activity to promote bone ingrowth and fusion, as well as radiolucency for facilitated post-operative monitoring. The fixation system may additionally carry one or more natural or synthetic therapeutic agents for further promoting bone ingrowth and fusion.

### BACKGROUND OF THE INVENTION

The spinal column is a highly complex system of bones and connective tissues that provides support for the body and protects the delicate spinal column and nerves. The spinal column includes a series of vertebrae stacked one atop the other, whereby each vertebral body includes a relatively strong bone portion forming the outside surface of the body (cortical) and a relatively weak bone portion forming the center of the body (cancellous). Situated between each vertebral body is an intervertebral disc that provides for cushioning and dampening of compressive forces applied to the spinal column. The vertebral canal containing the delicate spinal cords and nerves is located just posterior to the vertebral bodies.

Various types of spinal column disorders are known and include scoliosis (abnormal lateral curvature of the spine), kyphosis (abnormal forward curvature of the spine, usually in the thoracic spine), excess lordosis (abnormal backward curvature of the spine, usually in the lumbar spine), spondylolisthesis (forward displacement of one vertebra over another, usually in a lumbar or cervical spine) and other disorders caused by abnormalities, disease or trauma, such as ruptured or slipped discs, degenerative disc disease, fractured vertebra, and the like. Patients that suffer from such conditions usually experience extreme and debilitating pain as well as diminished nerve function.

The present invention involves a technique commonly referred to as spinal fixation whereby surgical implants are used for fusing together and/or mechanically immobilizing adjacent vertebrae of the spine. Spinal fixation may also be used to alter the alignment of the adjacent vertebrae relative to one another so as to alter the overall alignment of the spine. Such techniques have been used effectively to treat the above-described conditions and, in most cases, to relieve pain suffered by the patient. However, as will be set forth in more detail below, there are some disadvantages associated with current fixation devices.

One particular spinal fixation technique includes immobilizing the spine by using orthopedic rods, commonly referred to as spine rods, which run generally parallel to the spine. This is accomplished by exposing the spine posteriorly and fastening bone screws to the pedicles of the appropriate vertebrae. The pedicle screws are generally placed two per vertebra, one at each pedicle on either side of the spinous process, and serve as anchor points for the spine rods. Clamping elements adapted for receiving a spine rod therethrough are then used to join the spine rods to the screws. The aligning influence of the rods forces the spine to conform to a more desirable shape. In certain instances, the spine rods may be bent to achieve the desired curvature of the spinal column.

Another common spinal fixation technique is the use of a fixating plate with screws. A typical spinal fixation plate includes a relatively flat, rectangular plate having a plurality of apertures formed therein. A corresponding plurality of bone screws may be provided to secure the bone fixation plate to the vertebrae of the spine. These plates are generally attached to the anterior portion of the vertebral bodies. The screws may be rigidly constrained to the plate, or may be semi-constrained to allow for load sharing.

This invention relates generally to improvements in spinal fixation devices of the type designed for human implantation into adjacent spinal vertebrae, to maintain the vertebrae in substantially fixed spaced relation while promoting bone ingrowth and fusion therebetween. More particularly, this invention relates to screws and interconnecting rod or plate having an improved combination of enhanced mechanical strength together with osteoinductive and osteoconductive properties, in a device that additionally and beneficially provides visualization of bone growth for facilitated post-operative monitoring.

In typical posterior spinal fixation procedures, the space between the transverse processes of the two vertebral bodies are then usually filled with bone graft material, either autogenous bone material provided by the patient or allogenous bone material provided by a third party donor. In addition to this posterior lateral placement of fusion materials, such materials are often placed into the interbody space as well. The common method for a surgeon to analyze the growth of the bone in these areas is with the use of x-ray or magnetic resonance imaging (MRI).

In many anterior spinal fixation procedures, a graft is placed between the adjacent vertebrae in the interbody space. This graft is designed to enable or enhance bone growth between these vertebrae. The plate is then placed against the vertebral bodies, spanning the bone graft, and being directly adjacent to, if not touching, said bone graft. Again, the common method for a surgeon to analyze the growth of the bone in these areas is with the use of x-ray or magnetic resonance imaging.

Most commercially available spinal fixation systems are made from titanium alloys and have enjoyed clinical success as well as rapid and widespread use due to improved patient outcomes. However, traditional titanium-based implant devices exhibit radio-opaque characteristics, presenting difficulties in post-operative monitoring and evaluation of the fusion process using x-ray or fluoroscopic imaging. Radio-opacity presents a problem in that it does not allow structures located between the device and the imaging machine to be seen. Additionally, metallic implants cause scattering, or shadowing, and distortion of MRI's and CT's. These poor radiolucent properties can make it difficult, if not impossible to assess the bone growth using traditional means. In some cases, surgeons must use costly thin slice CT reconstruction to analyze the new bone growth. This is especially a problem for characterizing the bone growth between the transverse processes and in the interbody space, due to the titanium rod or plate being directly adjacent to the fusion material. Moreover, traditional titanium-based implant devices are primarily load bearing but are not osteoconductive, i.e., not conducive to direct and strong mechanical attachment to patient bone tissue, leading to potential micro-motion between the implant and the host bone, causing possible poor fusion, instability and bone resorption.

Another group of commercially available spinal fixation devices are made from various polymeric materials such as PEEK or polyurethane. However these devices have issues which make them difficult to use. One such problem is a lack of load bearing strength, which might lead to failure of the implant after surgery. Another issue is with intraoperative placement of the device, and postoperative radiographic analysis. Since these polymers are radiotransparent, they offer a solution to assessing bone growth via traditional radiographic imaging. However, this radiotransparency makes it extremely difficult for the surgeon to know where the device is located, both during and after implantation. Some devices utilize a radiographic marker to aid in this assessment, but exact location and orientation of the markers within the device still make it difficult for accurate assessment.

Autologous (patient) bone fusion has been used in the past and has a theoretically ideal mix of osteoconductive and osteoinductive properties. However, supply of autologous bone material is limited and significant complications are known to occur from bone harvesting. Moreover, the costs associated with harvesting autograft bone material are high, requiring two separate incisions, with the patient having to undergo more pain and recuperation due to the harvesting and implantation processes. Additionally, blood supply to the posterior lateral portion of the spine is generally low, meaning there is a lack of natural osteoinductive cells and growth factors, making it difficult to sustain bone growth in the area. This can cause pseudoarthrosis, which may lead to loosening or breakage of the implant and result in patient pain. It is also difficult to keep the autologous cancellous bone material in the proper placement between the transverse processes.

Ceramic materials provide potential alternative structures for use in spinal fusion implant devices. In this regard, monolithic ceramic constructs have been proposed, formed from conventional materials such as hydroxyapatitie (HAP) and/or tricalcium phosphate (TCP). See, for example, U.S. Pat. No. 6,037,519. However, while these ceramic materials may provide satisfactory osteoconductive and bio-active properties, they have not provided the mechanical strength necessary for the implant.

Thus, a significant need exists for further improvements in and to the design of spinal fixation devices, particularly to provide a high strength implant having high bone ingrowth and fusion characteristics, together with substantial radiolucency for effective and facilitated post-operative monitoring.

Hence, it is an object of the present invention to provide an improved spinal fixation device made from a bio-compatible, load bearing and imaging compatible material, with or without an open pore structure, which has radiolucency similar to that of the surrounding bone. Specifically, to provide a spinal fixation device with radiolucency that enables the surgeon to see the exact location and orientation of the implant utilizing traditional radiographic imaging, while still allowing for assessment of the bone growth in and around the device. It is also an object of the present invention to provide a substrate of adequate bio-mechanical strength for carrying biological agents which promote bone ingrowth, healing and fusion.

### SUMMARY OF THE INVENTION

In accordance with the invention, an improved spinal fixation system is provided for human implantation into a pair of adjacent vertebrae, to restore and maintain the spinal anatomy in a predetermined and substantially fixed spaced relation while promoting bone ingrowth and fusion. In this regard, the improved fixation device of the present invention is designed for use in addressing clinical problems indicated by surgical treatment of bone fractures, skeletal non-unions, weak bony tissue, degenerative disc disease, discogenic back pain, scoliosis (abnormal lateral curvature of the spine), kyphosis (abnormal forward curvature of the spine, usually in the thoracic spine), excess lordosis, and spondylolisthesis.

The improved fixation system comprises a bone screw and interconnecting rod or plate formed from a bio-compatible material composition having a relatively high bio-mechanical strength and load bearing capacity. These components may be porous, open-celled, or dense solid. A preferred material of the high strength substrate block comprises a ceramic material. The screws and rods may be porous, having a porosity of about 10% to about 80% by volume with uniformly distributed pores throughout and a pore size range of from about 5 to about 500 microns. When the component is porous, the porosity of the device is gradated from a first relatively low porosity region emulating or mimicking the porosity of cortical bone to a second relatively higher porosity region emulating or mimicking the porosity of cancellous bone. This structure mimicking of the porous properties of cancellous bone is called a bio-mimetic structure. In a second embodiment, the device is a dense solid comprised of a ceramic, metal or polymer material. This dense solid substrate would then be attached to a second highly porous, bio-mimetic region emulating or mimicking the porosity of cancellous bone. Preferably, the porous region would be integrally formed around or on the face of the substrate.

In the method where a dense, solid material is used as the substrate block, the block will be externally coated with a bio-active surface coating material selected for relatively high osteoconductive and bio-active properties, such as a hydroxyapatite or a calcium phosphate material. The porous portion is internally and externally coated with a bio-active surface coating material selected for relatively high osteoconductive and bio-active properties, such as a hydroxyapatite or a calcium phosphate material. The porous region, however, may be in and of itself a bio-active material selected for relatively high osteoconductive and bio-active properties, such as a hydroxyapatite or a calcium phosphate material.

The thus-formed fixation device can be made in a variety of shapes and sizes to suit different specific implantation requirements. Preferred shapes include a rod or plate with a lordotic curvature. This rod has a dense inner cylinder of high strength for supporting spinal loading. The dense inner cylinder is surrounded along its axis by a structure of open porosity. The plate component is made of a dense body of high strength for receiving the screws and supporting load. The face of the plate which lies adjacent to the vertebral body is covered with a structure of open porosity. In turn, the porous structure has osteoconductive materials coating throughout the pores. This preferred embodiment aids in the fusion along the rod or plate, which is placed between transverse processes or adjacent to the interbody space. Additional preferred shapes include that of a bone screw. The bone screw is comprised of a dense substrate of high strength for spinal loading. Portions of the threaded shank of the screw are surrounding by a structure of open porosity. In turn, the porous structure has osteoconductive materials coating throughout the pores. This enables bone growth into the screw itself, thereby aiding in the fixation of the device to the vertebral body.

The resultant spinal fixation device exhibits relatively high mechanical strength for load bearing support, while additionally and desirably providing high osteoconductive and osteoinductive properties to achieve enhanced bone ingrowth and fusion. Importantly, these desirable characteristics are achieved in a structure which is substantially radiolucent so that the implant does not interfere with post-operative radiographic monitoring of the fusion process.

In accordance with a further aspect of the invention, the spinal fixation device may additionally carry one or more therapeutic agents for achieving further enhanced bone fusion and ingrowth. Such therapeutic agents may include natural or synthetic therapeutic agents such as bone morphogenic proteins (BMPs), growth factors, bone marrow aspirate, stem cells, progenitor cells, antibiotics, or other osteoconductive, osteoinductive, osteogenic, bio-active, or any other fusion enhancing material or beneficial therapeutic agent.

Other features and advantages of the invention will become more apparent from the following detailed description, taken in conjunction with the accompanying drawings which illustrate, by way of example, the principles of the invention.

### BRIEF DESCRIPTION OF THE DRAWINGS

The accompanying drawings illustrate the invention. In such drawings:

FIG. 1 is a perspective view depicting the spinal fixation device;

FIG. 2 is a perspective view of the spinal fixation device of FIG. 1 shown implanted into the spine;

FIG. 3 is an exploded perspective view of the device shown in FIG. 1;

FIG. 4 is an example of the rod in FIG. 1, without the porous structure surrounding it;

FIG. 5 is closeup view of threads of another preferred embodiment of the screw;

FIG. 6 is a perspective view of still another preferred embodiment of the screw;

FIG. 7 is a perspective view of another preferred embodiment of the rod;

FIG. 8 is a perspective view of another preferred embodiment of the spinal fixation device comprised of the screw of FIG. 6 and the rod of FIG. 7;

FIG. 9 is a perspective view of still another preferred embodiment of the screw with a porous structure around the head;

FIG. 10 is a perspective view of yet another preferred embodiment of the spinal fixation device depicting screws and a plate;

FIG. 11 is another view of the device in FIG. 10 illustrating the porous structure;

FIG. 12 is still another preferred embodiment of the spinal fixation involving radiolucent screws and rod;

FIG. 13 is yet another preferred embodiment of the spinal fixation involving radiolucent screws and plate.

### DETAILED DESCRIPTION OF THE PREFERRED EMBODIMENTS

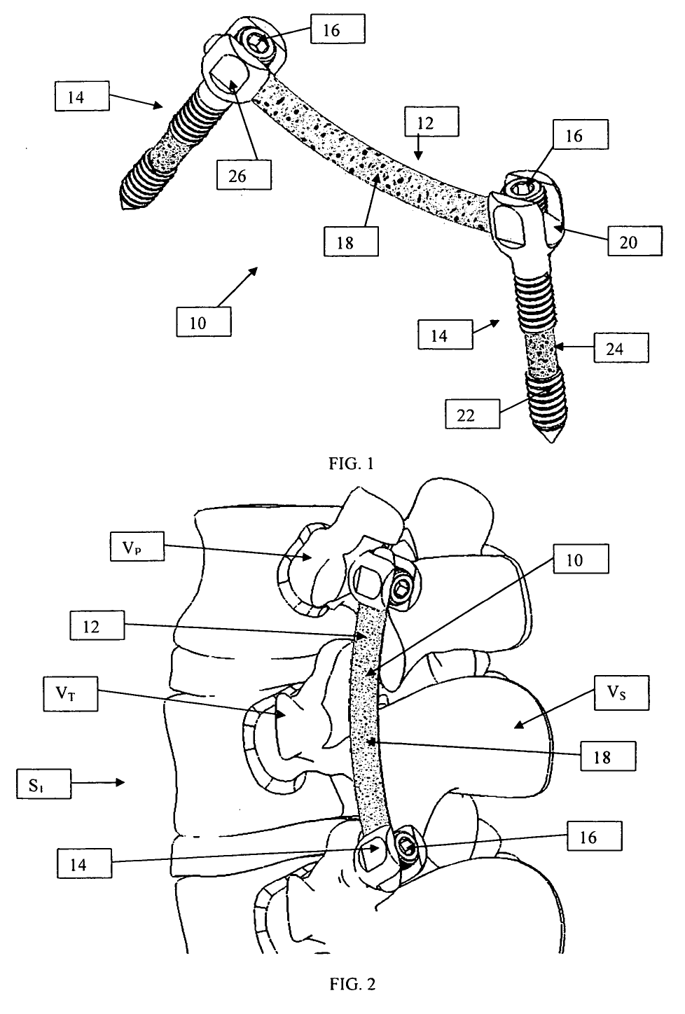

As shown in the exemplary drawings, an osteoconductive spinal fixation device referred to generally in FIGS. 1-3 by the reference numeral 10 is provided for attachment to at least a pair of adjacent patient bones such as spinal vertebrae S₁ (FIG. 2) to maintain the skeletal structures in spaced relation while promoting bone ingrowth and fusion. In general, the improved fixation device 10 comprises a bio-compatible support structure such as the illustrative rod 12 having a dense substrate 34 (FIG. 4) providing a strong mechanical load bearing structure and a porous construction 18 to define an open lattice conducive to bone ingrowth and fusion. The preferred embodiment is manufactured from a high strength ceramic material, allowing for load carrying abilities, as well as substantial radiolucency and non-magnetic characteristics. This open-celled construction 18 is coated internally and externally with a biologic coating selected for relatively high osteoconductive and bio-active properties, whereby the coated construction 18 provides a scaffold conducive to cell attachment and proliferation to promote bone ingrowth and fusion attachment. The substrate may also carry one or more selected therapeutic agents suitable for bone repair, augmentation and other orthopedic uses. Said rod 12 is connected between two bio-compatible bone screws 14 which are in turn anchored to the skeletal structure S₁.

The bone screw 14 is comprised of a dense body or shank which has at least one threaded portion or segment 22 for engaging and anchoring to bone. The preferred embodiment is manufactured from a high strength ceramic material, allowing for load carrying abilities, as well as substantial radiolucency and non-magnetic characteristics. Proximal to the threaded portion 22 is a head section 26 that is design for receiving the rod 12. The portion of the rod being received into the head section 26 of the screw 14 is that of the dense, strong mechanical portion located at the end 20 of the rod. This dense end 20 of the rod is fixated to the screw head 26 by means of a locking screw 16.

Portions of the bone screw 14 disposed axially adjacent to and preferably axially between thread segments 22 are of a porous construction 24 to define another open lattice conducive to bone ingrowth and fusion. This open-celled construction 24 is coated internally and externally with a biologic coating selected for relatively high osteoconductive and bio-active properties, whereby the coated construction 24 provides a scaffold conducive to cell attachment and proliferation to promote bone ingrowth and fusion attachment. This aids in the fixation of the bone screw 14 to the host skeletal structure S₁. The substrate may also carry one or more selected therapeutic agents suitable for bone repair, augmentation and other orthopedic uses.

The resultant illustrative fixation device 10 exhibits relatively high bio-mechanical strength similar to the load bearing characteristics. In addition, the fixation device 10 exhibits relatively high osteoconductive and bio-active characteristics attributable primarily to the surface coating, again similar to natural bone. Importantly, the fixation device 10 is also substantially radiolucent and non-magnetic, so that the device does not interfere with post-operative radiological or other imaging methods of analysis of bone ingrowth and fusion.

FIG. 2 shows the preferred fixation device 10 attached to a skeletal structure S₁, specifically the vertebrae of the lumbar spine. Each of the bone screws 14 are anchored into one of the pedicles V_{P} of the spine S₁. It is inside of the pedicle V_{P} that the porous portion 24 of the screw is intended to aid in the bone growth and fixation of the host bone S₁ to the screw 14. In order to stabilize the spine S₁, the bone screws 14 are connected together via the rod component 12 and the locking screws 16. The rod component 12 runs adjacent to the axis of the spine, lateral of the spinous processes V_{S}, and medial of the transverse processes V_{T}. It is in this area that autologous bone is generally placed in an attempt to fuse the adjacent vertebrae together. Additionally, this is the area in which the rod 12 has its integrated porous structure 18. Since the porous structure 18 of the rod exhibits relatively high osteoconductive and bi-active characteristics attributable primarily to the surface coating, it aids in the promotion of bone growth and fusion around it. Furthermore, due to the open cell porosity of this structure, it encourages bone growth into the rod 12 component itself, thereby creating further stability and fixation throughout the spinal segments S₁. Importantly, the fixation device 10 is also substantially radiolucent and non-magnetic, so that the device does not interfere with post-operative radiological or other imaging methods of analysis of bone growth and fusion.

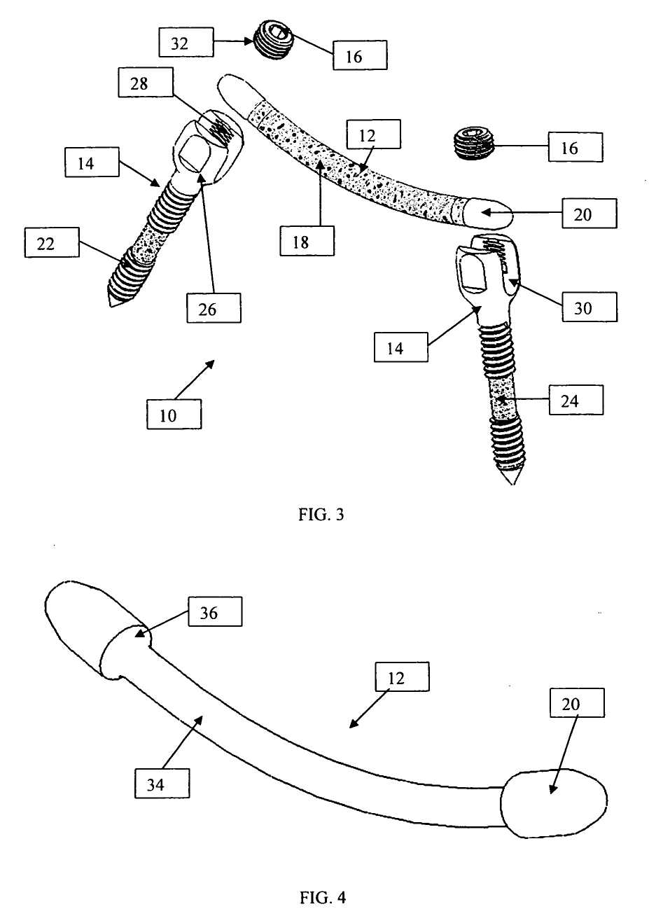

FIG. 3 depicts an exploded version of the device 10 discussed earlier. This illustrates the basic method with which the system is assembled. The head 26 of each bone screw 14 has a receiving slot 30 for the rod 12 to seat. The ends 20 of the rod 12, being of substantially dense material to allow for greater mechanical strength, are introduced into the receiving cavities or slots 30 of the respective bone screws 14. The rod 12 is fixated against the respective screw head 26 by a locking screw 16 which is seated atop of the ends of the rod 20. The threads 32 of the locking screw 16 engage threads 28 on the interior of the bone screw head 26.

The rod 12 is shown in FIG. 4 with the porous structure 18 removed to better illustrate the design of the dense portion only. Extending between the dense ends 20 of the rod is a dense, load bearing substrate 34 that supports and maintains the appropriate spacing of the spine. There is a transition point 36 between the end 20 and the substrate 34 of the rod is designed to reduce stresses.

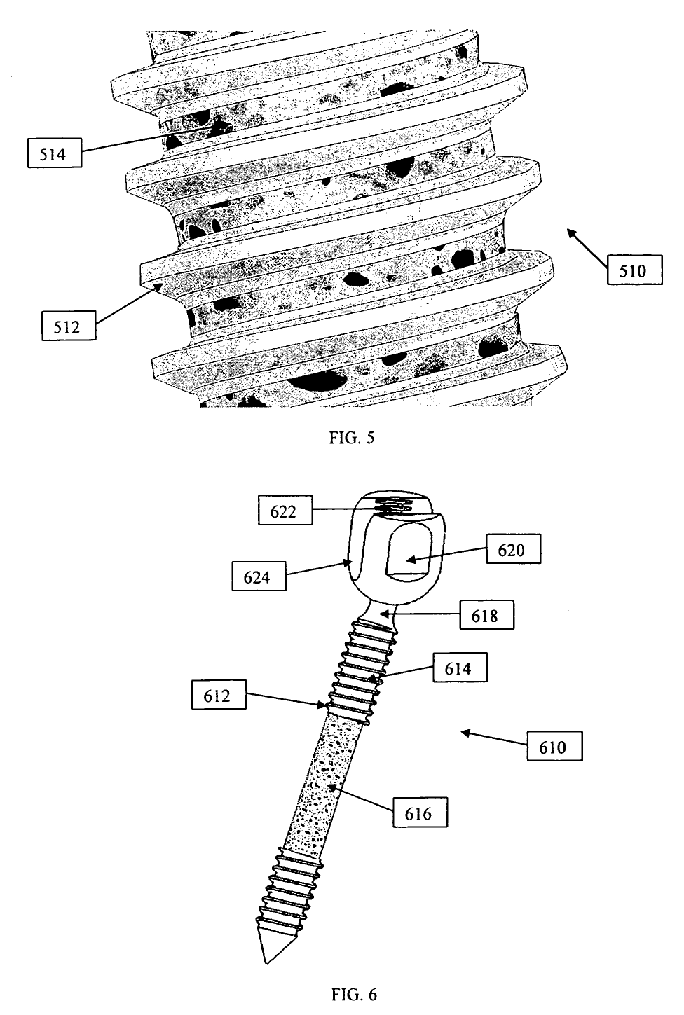

FIG. 5 illustrates another preferred embodiment of the bone screw construction. This embodiment of the bone screw 510 is similar to the version described earlier and referenced by numeral 14 in FIGS. 1-3. However, the bone screw 510 has an additional porous structure 514 located along the portion of the shaft which contains the bone engaging threads 512. These bone threads 512 are constructed of generally dense material of high mechanical strength enabling them to cut through the bone as the screw advances into the host bone. Additionally, the high strength dense threads 512 must be strong enough to resist pulling out of the bone when such loading and stresses would present such an event. However, along the minor diameter of the thread form is located a spiral-shaped porous structure 514 which wraps around the body or shank of the screw for the entire thread length. This allows for a continuous thread form to extend along the screw length from the point to the head. It also creates an unbroken porous ingrowth structure along that same length of the screw 510.

Still another preferred embodiment of the bone screw 610 is illustrated in FIGS. 6 and 8. This particular embodiment of the bone screw 610 is composed two components, the screw body 612 and a housing 620. The screw body component 612 has a threaded portion 614 for engaging and anchoring into the host bone. The threaded portion is constructed of generally dense material of high mechanical strength enabling them the cut through the bone as the screw advances into the host bone. Additionally, the high strength dense threads 614 must be strong enough to resist pulling out of the bone when such loading and stresses would present such an event. Along said threaded portion is a porous structure 616 which exhibits relatively high osteoconductive and bio-active characteristics attributable primarily to the surface coating, it aids in the promotion of bone growth and fusion around it. Furthermore, due to the open cell porosity of this structure, it encourages bone growth into the screw 612 component itself, thereby creating further stability and fixation to the host bone. The head 618 of the screw body 612 is captured within the housing 620, where it is allowed to articulate. This enables the surgeon greater flexibility for inserting the bone screw into the bone, and subsequently attaching the rod to said screw. The housing 620 of the bone screw has a rod receiving slot 624 and an internally threaded portion 622 for receiving a locking screw.

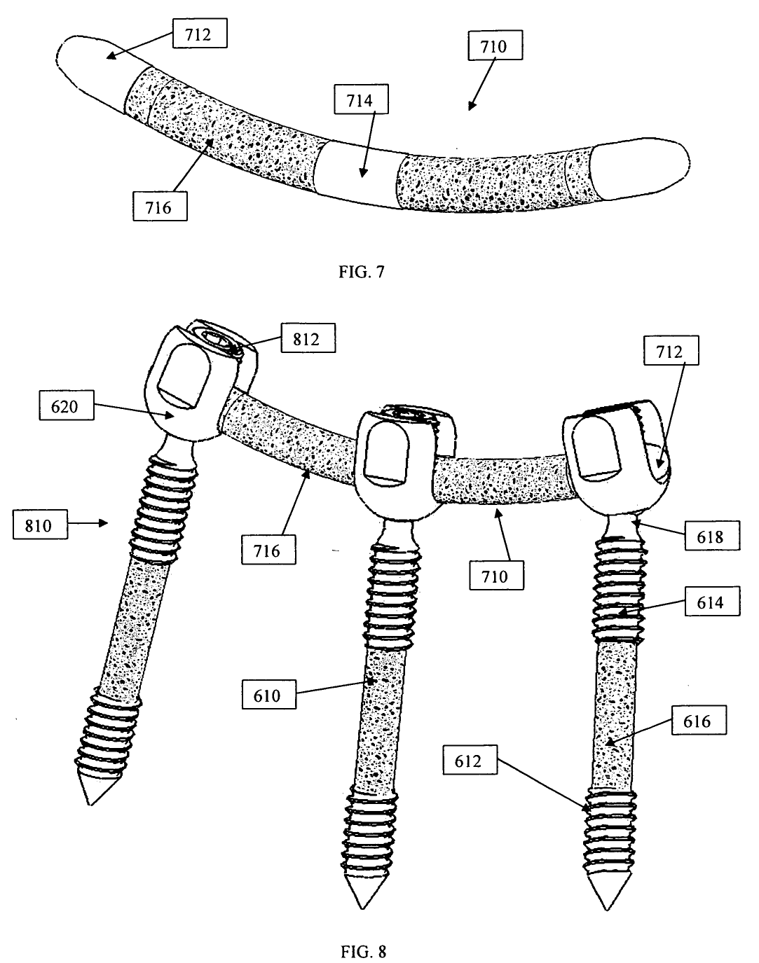

FIGS. 7-8 depict another preferred embodiment of the rod component of the improved fixation device. The rod 710 is comprised of a dense substrate providing a strong mechanical load bearing structure and a porous construction 716 to define an open lattice conducive to bone ingrowth and fusion. The rod has multiple attachment points for interfacing with the screw component 610. These attachment points are located at the ends 712 of the rod, as well as the middle 714 of the rod. The multiple attachment points allow for more than two screws to be interconnected by the rod 710, and therefore more than two bone segments to be fixated and fused by the improved fixation device. Located between each attachment point and along the axis of the rod 710 is an open-celled porous structure 716. This open-celled construction 716 is coated internally and externally with a biologic coating selected for relatively high osteoconductive and bio-active properties, whereby the coated construction 716 provides a scaffold conducive to cell attachment and proliferation to promote bone ingrowth and fusion attachment. The substrate may also carry one or more selected therapeutic agents suitable for bone repair, augmentation and other orthopedic uses.

FIG. 8 shows the basic method of assembly of the preferred embodiments as described earlier in FIGS. 6-7. The housing 620 of the bone screw 610 has a receiving slot 624 for the attachment points 712 and 714 of the rod 710 to seat. The attachment points 712 and 714 of the rod 710, being of substantially dense material to allow for greater mechanical strength, are introduced into the receiving slot 624 of the bone screw housing 620. This allows the porous portion 716 of said rod to be exposed to the host to enable bone growth and fusion. The rod 710 is fixated against the bone screw housing 620 by a locking screw 812 which is seated atop of the attachment points 712 and 714 of the rod 710. The threads of the locking screw 812 engage threads 622 on the interior of the bone screw housing 620. The screw body 612 is allowed to articulate within the housing 620 until final tightening of the locking screw 812.

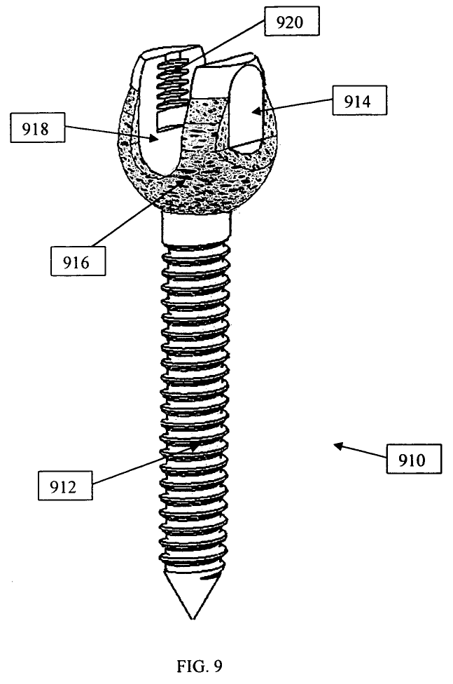

FIG. 9 depicts still another preferred embodiment of the bone screw component of the improved fixation device. The bone screw 910 is composed of a bone thread portion, for engaging and anchoring to the host bone, and a head portion 914 for receiving and attaching to a rod component. The head portion 914 has a receiving slot 918 for mating with the attachment points of the rod component. Additionally, the head has an internally threaded portion 920 for receiving a locking screw, which fixates the rod to the bone screw 910. These portions of the bone screw 910 are of generally high strength, dense material for load carrying and bone cutting properties. However, around the exterior of the head 914 is an open celled, porous structure 916. This open-celled construction 916 is coated internally and externally with a biologic coating selected for relatively high osteoconductive and bio-active properties, whereby the coated construction 916 provides a scaffold conducive to cell attachment and proliferation to promote bone ingrowth and fusion attachment. The substrate may also carry one or more selected therapeutic agents suitable for bone repair, augmentation and other orthopedic uses.

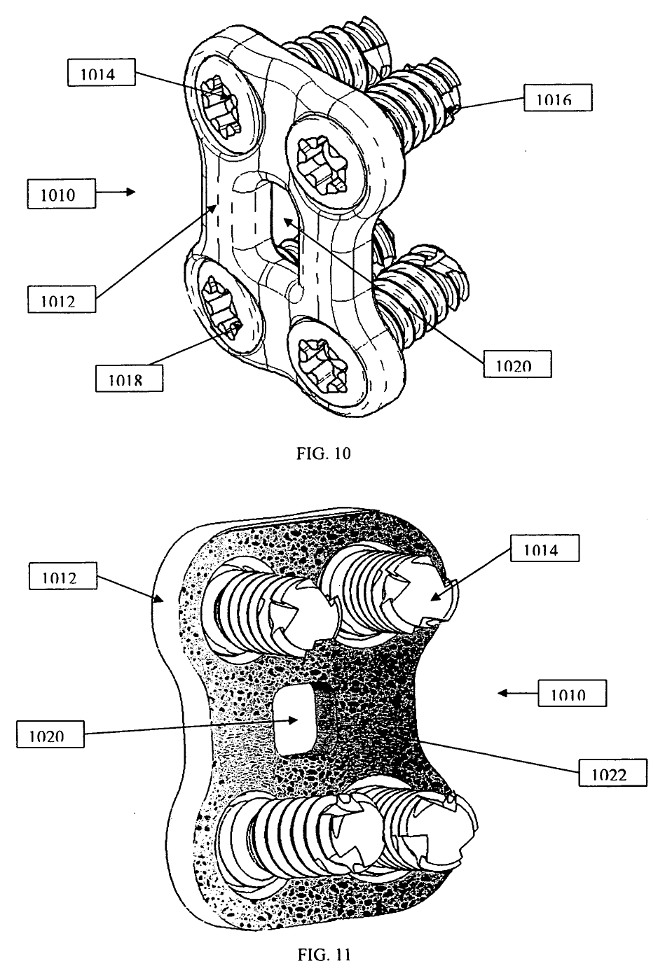

An osteoconductive spinal fixation device referred to generally in FIGS. 10-11 by the reference numeral 1010 is provided for attachment to at least a pair of adjacent patient bones such as spinal vertebrae to maintain the skeletal structures in spaced relation while promoting bone ingrowth and fusion. In general, the improved fixation device 1010 comprises an alternative bio-compatible support structure such as the illustrative bio-compatible plate 1012 having a dense substrate providing a strong mechanical load bearing structure and a porous construction 1022 to define an open lattice conducive to bone ingrowth and fusion. The preferred embodiment is manufactured from a high strength ceramic material, allowing for load carrying abilities, as well as substantial radiolucency and non-magnetic characteristics. This open-celled construction 1022 is coated internally and externally with a biologic coating selected for relatively high osteoconductive and bio-active properties, whereby the coated construction 1022 provides a scaffold conducive to cell attachment and proliferation to promote bone ingrowth and fusion attachment. The substrate may also carry one or more selected therapeutic agents suitable for bone repair, augmentation and other orthopedic uses. Said plate 1012 is connected between a plurality of bio-compatible bone screws 1014 which are in turn anchored to the skeletal structure.

Each bone screw 1014 is comprised of a dense body which has a threaded portion 1016 for engaging and anchoring to bone. The preferred embodiment is manufactured from a high strength ceramic material, allowing for load carrying abilities, as well as substantial radiolucency and non-magnetic characteristics. The plate component 1012 has apertures for receiving the head section 1018 of said screw 1014, allowing the threaded portion 1016 to pass through the aperture. The portion of the plate receiving the head of the screw 1018 is that of the dense, strong mechanical portion. To aid in direct visualization intraoperatively, the plate 1012 may have a window 1020 to view the bone graft.

Portions of the bone screw 1014 may also be of porous construction, as demonstrated previously in various embodiments depicted in FIGS. 5-6 to define another open lattice conducive to bone ingrowth and fusion. This open-celled construction is coated internally and externally with a biologic coating selected for relatively high osteoconductive and bio-active properties, whereby the coated construction provides a scaffold conducive to cell attachment and proliferation to promote bone ingrowth and fusion attachment. This aids in the fixation of the bone screw 1014 to the host skeletal structure. The substrate may also carry one or more selected therapeutic agents suitable for bone repair, augmentation and other orthopedic uses. In an alternate embodiment, this open-celled construction may be coated internally and externally with a bone cement, whereby the coated construction provides a secure attachment to osteoporotic bone.

The resultant illustrative fixation device 1010 exhibits relatively high bio-mechanical strength similar to the load bearing characteristics. In addition, the fixation device 1010 exhibits relatively high osteoconductive and bio-active characteristics attributable primarily to the surface coating, again similar to natural bone. Importantly, the fixation device 1010 is also substantially radiolucent and non-magnetic, so that the device does not interfere with post-operative radiological or other imaging methods of analysis of bone ingrowth and fusion.

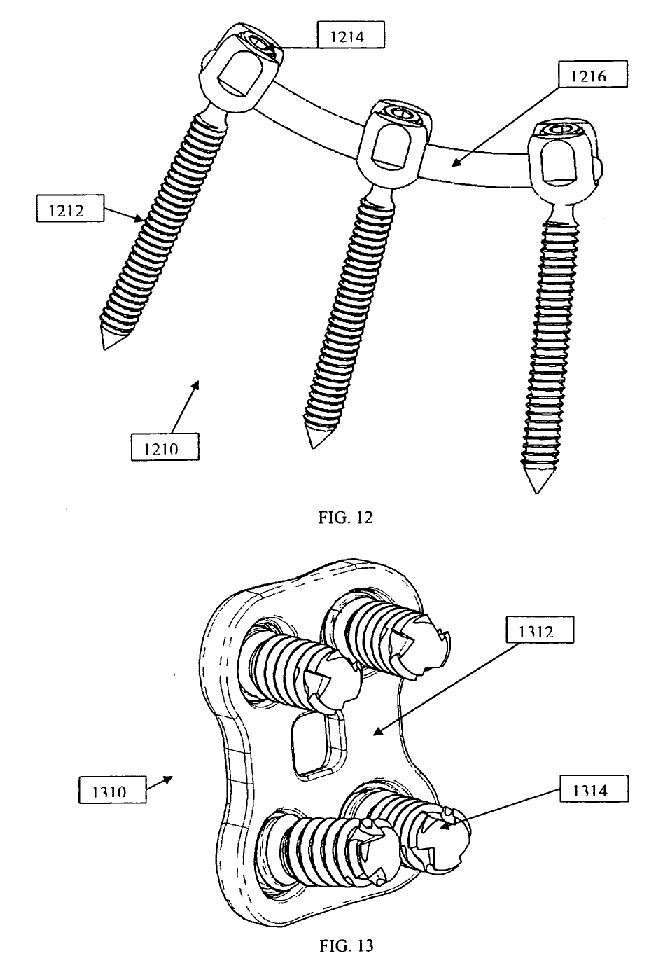

The spinal fixation devices depicted in FIGS. 12-13 illustrate a preferred embodiment of the present invention utilizing substantially radiolucent materials without the presence of a porous structure. The device 1210 in FIG. 12 shows a pedicle screw and rod system constructed of substantially radiolucent materials. The system consists of two or more screws 1212 which are designed to receive a rod 1216 connecting between the screws. The rod 1216 is secured in place by use of a locking screw 1214. The device 1310 depicting FIG. 13 shows a plate and screw system which is also constructed of substantially radiolucent materials. This systems consists of a plate 1312 with apertures designed for receiving a plurality of screws 1314. These embodiments, while not having a porous, osteoconductive structure, are still advantageous to the prior art in that it is substantially radiolucent and non-magnetic so that the device does not interfere with post-operative radiological or other imaging methods of analysis of bone ingrowth and fusion. Furthermore, it is intended that certain components of these, and the previous, embodiments may be constructed of substantially radiolucent and non-magnetic materials, such as silicon nitride, alumina or the like, while other components of the same system may be constructed of radio-opaque components, such as titanium.

The improved fixation device of the present invention thus comprises an open-celled porous structure which is coated with a bio-active surface coating, and has the strength required for the weight bearing capacity required of a fusion device. The capability of being infused with the appropriate biologic coating agent imparts desirable osteoconductive and bio-active properties to the device for enhanced interbody bone ingrowth and fusion, without detracting from essential load bearing characteristics. The radiolucent or non-magnetic characteristics of the improved device beneficially accommodate post-operative radiological or other diagnostic imaging examination to monitor the bone ingrowth and fusion progress, substantially without undesirable radio-shadowing. In addition to these benefits, the present invention is easy to manufacture in a cost competitive manner. The invention thus provides a substantial improvement in addressing clinical problems indicated for surgical treatment of scoliosis (abnormal lateral curvature of the spine), kyphosis (abnormal forward curvature of the spine, usually in the thoracic spine), excess lordosis (abnormal backward curvature of the spine, usually in the lumbar spine), spondylolisthesis (forward displacement of one vertebra over another, usually in a lumbar or cervical spine) and other disorders caused by abnormalities, disease or trauma, such as ruptured or slipped discs, degenerative disc disease, fractured vertebra, and the like.

The fixation device of the present invention provides at least the following benefits over the prior art:

[a] a porous osteoconductive scaffold for enhanced fusion rates;

[b] a bio-mimetic load bearing superstructure providing appropriate stress transmission without fatigue failure;

[c] a pore structure and size suitable for ingrowth and vascularization, [d] the ability to absorb and retain an osteoinductive agent such as autologous bone marrow aspirate or BMPs;

[e] bio-inert and bio-compatible with adjacent tissue and selected for ease of resorption;

[f] radiolucent and MRI compatible;

[g] fabricatable and machinable into various shapes;

[h] sterilizable; and

[i] low manufacturing cost.

A variety of further modifications and improvements in and to the fixation device of the present invention will be apparent to those persons skilled in the art. In this regard, it will be recognized and understood that the fixation device can be formed in the size and shape of a plate with screws for implantation into a bone regeneration/ingrowth site. Accordingly, no limitation on the invention is intended by way of the foregoing description and accompanying drawings, except as set forth in the appended claims.

What is claimed is:

1. A device for the stabilization of one or more bone segments, comprising:
at least one bone screw for attachment to the one or more bone segments; and
a bio-compatible support structure carried by said at least one bone screw, whereby said at least one bone screw retains said bio-compatible support structure relative to the one or more bone segments;
at least one of said bone screw and said bio-compatible support structure including a first region of relatively high strength and a second region of porous form corresponding substantially with natural cancellous bone, said second region being disposed on at least a portion of the exterior of said one of said at least one bone screw and said bio-compatible support structure for bone ingrowth and fusion attachment with the adjacent one or more bone segments.

2. The stabilization device of claim 1 further including means for interconnecting said at least one bone screw with said bio-compatible support structure.

3. The stabilization device of claim 1 wherein said at least one bone screw comprises a plurality of bone screws for attachment to the at least one or more bone segments.

4. The stabilization device of claim 1 wherein said at least one bone screw includes said first region of relatively high strength and said second region of porous form corresponding substantially with natural cancellous bone.

5. The stabilization device of claim 4 wherein said at least one bone screw includes an elongated shank, said first region comprising an externally exposed thread on said shank, and said second region comprising a spiral-shaped porous structure extending about said shank between said exposed thread.

6. The stabilization device of claim 1 wherein said at least one bone screw includes an elongated shank, said first region comprising at least one externally exposed thread segment formed on said shank, and said second region being formed on said shank axially adjacent said thread segment.

7. The stabilization device of claim 1 wherein said at least one bone screw includes an elongated shank, said first region comprising at least two externally exposed thread segments formed on said shank, and said second region being formed on said shank axially between said thread segments.

8. The stabilization device of claim 1 wherein said at least one bone screw include a head, and further including means for connection of said head to said bio-compatible support structure.

9. The stabilization device of claim 8 wherein said bio-compatible support structure comprises an elongated rod having at least one first region externally exposed for connection to said at least one bone screw.

10. The stabilization device of claim 9 wherein said at least one bone screw head defines a cavity for seated reception of said rod first region, said connection means further including means for retaining said rod first region within said cavity.

11. The stabilization device of claim 10 wherein said retaining means comprises a lock screw.

12. The stabilization device of claim 9 wherein said connection means comprises a housing movably carried by said head, and means for retaining said rod first region relative to said housing.

13. The stabilization device of claim 9 wherein said rod has a plurality of externally exposed first regions, said connecting means including means for retaining said plurality of externally exposed first regions within respective head cavities of a corresponding plurality of said bone screws.

14. The stabilization device of claim 8 wherein said bone screw head further includes said second region formed thereon.

15. The stabilization device of claim 1 wherein said bio-compatible support structure includes said first region of relatively high strength and said second region of porous form corresponding substantially with natural cancellous bone.

16. The stabilization device of claim 1 wherein said at least one bone screw and said bio-compatible support structure both include said first region of relatively high strength and said second region of porous form corresponding substantially with natural cancellous bone.

17. The stabilization device of claim 1 wherein said bio-compatible support structure comprises a rod.

18. The stabilization device of claim 1 wherein said bio-compatible support structure comprises a plate.

19. The stabilization device of claim 1 wherein said at least one bone screw is formed from a ceramic material selected from the group consisting essentially of silicon nitride, alumina, zirconia, zirconia toughened alumina, hydroxyapatite, calcium phosphate, and compositions thereof.

20. The stabilization device of claim 1 wherein said bio-compatible support structure is formed from a ceramic material selected from the group consisting essentially of silicon nitride, alumina, zirconia, zirconia toughened alumina, hydroxyapatite, calcium phosphate, and compositions thereof.

21. The stabilization device of claim 1 wherein said at least one bone screw and said bio-compatible support structure are both formed from a ceramic material selected from the group consisting essentially of silicon nitride, alumina, zirconia, zirconia toughened alumina, hydroxyapatite, calcium phosphate, and compositions thereof.

22. The stabilization device of claim 1 wherein at least one of said bone screw and said bio-compatible support structure further includes a biologic surface coating applied thereto, said surface coating having osteoconductive and bio-active properties to promote bone ingrowth and fusion attachment with the adjacent one or more bone segments.

23. The stabilization device of claim 22 wherein said biologic surface coating is applied internally and externally to said second region.

24. The stabilization device of claim 22 wherein said biologic surface coating is applied externally to said first region.

25. The stabilization device of claim 22 wherein said biologic surface coating comprises a material selected from the group consisting essentially of hydroxyapatite and calcium phosphate materials.

26. The stabilization device of claim 22 wherein said biologic surface coating comprises a partially or fully amorphous bio-active material including a glass and bio-active calcium compound.

27. The stabilization device of claim 22 wherein said bio-active surface coating comprises an organic coating material.

28. The stabilization device of claim 27 wherein said organic coating material is selected from the group consisting of autologous bone marrow aspirates, bone morphogenic proteins, growth factors and progenitor cells, and mixtures thereof.

29. The stabilization device of claim 28 wherein said progenitor cells include mesenchymal stem cells, hematopoietic cells, and embryonic stem cells.

30. The stabilization device of claim 1 wherein said first region is relatively non-resorbable or resorbable at a rate substantially less than said second region.

31. The stabilization device of claim 1 wherein said first region and said second region have a porosity ranging from about 0% to about 80% by volume, and further wherein the pore size ranges from about 1 micron to about 1,500 microns.

32. The stabilization device of claim 31 wherein said first region has a porosity ranging from about 0% to about 50% by volume, and wherein the pore sizes range from about 1 micron to about 500 microns.

33. The stabilization device of claim 31 wherein said second region has a porosity ranging from about 30% to about 80% by volume, and wherein the pore sizes range from about 100 microns to about 1000 microns.

34. The stabilization device of claim 1 further including a porosity gradient between said first and second regions.

35. The stabilization device of claim 1 wherein said second region circumferentially surrounds said first region.

36. The stabilization device of claim 1 wherein said first region comprises at least one structural load bearing strut extending through said at least one of said bone screw and said bio-compatible support structure, and further wherein said second region defines an extended externally exposed surface area.

37. The stabilization device of claim 1 wherein said first region is substantially radiolucent.

38. The stabilization device of claim 1 wherein said second region is substantially radiolucent.

39. The stabilization device of claim 1 wherein at least one of said bone screw and said bio-compatible support structure further includes a therapeutic agent carried thereby.

40. The stabilization device of claim 1 wherein said at least one bone screw is substantially radiolucent.

41. The stabilization device of claim 1 wherein said bio-compatible support structure is substantially radiolucent.

42. A device for the stabilization of one or more bone segments, comprising:
at least one bone screw for attachment to the one or more bone segments; and
a bio-compatible support structure carried by said at least one bone screw, whereby said at least one bone screw retains said bio-compatible support structure relative to the one or more bone segments;
at least one of said bone screw and said bio-compatible support structure being formed from a substantially radiolucent material.

43. The stabilization device of claim 42 further including means for interconnecting said at least one bone screw with said bio-compatible support structure.

44. The stabilization device of claim 42 wherein said substantially radiolucent material includes a first region of relatively high strength and a second region of porous form corresponding substantially with natural cancellous bone, said second region being disposed on at least a portion of the exterior of said at least one bone screw and said bio-compatible support structure for bone ingrowth and fusion attachment with the adjacent one or more bone segments.

45. The stabilization device of claim 42 wherein the said screw is substantially radiolucent.

46. The stabilization device of claim 43 wherein said bio-compatible support structure is substantially radiolucent.

47. The stabilization device of claim 42 wherein said bio-compatible support structure is substantially radiolucent.

48. The stabilization device of claim 42 wherein said bio-compatible support structure comprises a rod.

49. The stabilization device of claim 42 wherein said bio-compatible support structure comprises a plate.

50. A device for the stabilization of one or more bone segments, comprising:
a bone screw having a head and a threaded shank for attachment to the one or more bone segments;
said bone screw defining a first region of relatively high strength and a second region of porous form corresponding substantially with natural cancellous bone, said second region being disposed on at least a portion of the exterior of said bone screw for bone ingrowth and fusion attachment with the adjacent one or more bone segments.

51. The stabilization device of claim 50 wherein said bone screw includes an elongated shank, said first region comprising an externally exposed thread on said shank, and said second region comprising a spiral-shaped porous structure extending about said shank between said exposed thread.

52. The stabilization device of claim 50 wherein said bone screw includes an elongated shank, said first region comprising at least one externally exposed thread segment formed on said shank, and said second region being formed on said shank axially adjacent said thread segment.

53. The stabilization device of claim 50 wherein said bone screw includes an elongated shank, said first region comprising at least one two externally exposed thread segments formed on said shank, and said second region being formed on said shank axially between said thread segments.

54. The stabilization device of claim 50 further including a bio-compatible rod defining a first region of relatively high strength and a second region of porous form corresponding substantially with natural cancellous bone, said rod first region being externally exposed for connection to said bone screw, and said rod second region being disposed for bone ingrowth and fusion attachment with the adjacent one or more bone segments.

55. The stabilization device of claim 54 wherein said bone screw includes a head defining a cavity for seated reception of said rod first region, and further including connection means for retaining said rod first region within said cavity.

56. The stabilization device of claim 55 wherein said retaining means comprises a lock screw.

57. The stabilization device of claim 55 wherein said connection means comprises a housing movably carried by said head, and means for retaining said rod first region within said housing.

59. The stabilization device of claim 55 wherein said bone screw head further includes said second region formed thereon.

60. The stabilization device of claim 50 further including a bio-compatible plate defining a first region of relatively high strength and a second region of porous form corresponding substantially with natural cancellous bone, said plate first region being externally exposed for connection to said bone screw, and said plate second region being disposed for bone ingrowth and fusion attachment with the adjacent one or more bone segments.
